(19) United States
(12) Patent Application Publication
(10) Pub. No.: US 2022/0226506 A1
(43) Pub. Date: Jul. 21, 2022

(54) EXPRESSION CONSTRUCTS FOR THE GENETIC MODIFICATION OF CELLS

(71) Applicant: Deutsches Krebsforschungszentrum, Heidelberg (DE)

(72) Inventors: Matthias Bozza, Heidelberg (DE); Richard Harbottle, Heidelberg (DE)

(21) Appl. No.: 17/439,921

(22) PCT Filed: Mar. 17, 2020
(86) PCT No.: PCT/EP2020/057234

§ 371 Date: Sep. 16, 2021

(30) Foreign Application Priority Data
Mar. 18, 2019 (EP) 19163509.3

(51) Int. Cl. A61K 48/00; C12N 15/90; C12N 5/071; C12N 15/62; C12N 15/86; C12N 15/67

(52) U.S. Cl. CPC A61K 48/0066; C12N 15/907; C12N 5/0687; C12N 15/625; C12N 2830/46; C12N 15/67; C12N 2740/15043; C12N 2830/42; C12N 15/86

(57) ABSTRACT

The present invention relates to a polynucleotide comprising at least one promoter, at least one expressible construct, and an S/MAR element, wherein said polynucleotide is an integration construct or a non-integrative vector construct, wherein said S/MAR element is located downstream of said promoter and of said expressible construct, and wherein said S/MAR element is flanked by a splice donor and a splice acceptor. The present invention also relates to a composition and a host cell comprising said polynucleotide, as well as to uses and methods related thereto.

The present invention relates to polynucleotide comprising at least one promoter, at least one expressible construct, and an S/MAR element, wherein said polynucleotide is an integration construct or a non-integrative vector construct, wherein said S/MAR element is located downstream of said promoter and of said expressible construct, and wherein said S/MAR element is flanked by a splice donor and a splice acceptor. The present invention also relates to a composition and a host cell comprising said polynucleotide, as well as to uses and methods related thereto.

Genetic modification of cells is used routinely in modern cell culture for scientific purposes. However, use of corresponding techniques in treatment of inherited diseases caused by mutations of genes, while being highly desirable, still is hampered by the problem that methods available usually only provide transient modification, such as transient transfection protocols, whereas methods providing stable modification of cells usually rely on integration of the transgene into the genome of the host cell. Integration of a transgene, however, even if targeted to a specific locus, bears the risk of inducing a deleterious mutation, which may lead e.g. to cancer as a side effect of treatment.

Scaffold/matrix attachment regions (S/MARs), which are also known as scaffold-attachment regions (SARs) or matrix-associated regions (MARs) are known as sequences in the genome of eukaryotic organisms mediating attachment of the nuclear matrix. Moreover, S/MAR sequences were found to have insulator properties, preventing extension of a condensed chromatin domain into a transcriptionally active region and the interaction of a distal enhancer with a promoter (Yusufzai & Felsenfeld (2004), PNAS 101(23), 8620). The S/MARS are AT-rich sequences, and some AT-rich motifs were found to be further enriched (Liebeich et al. (2002), NAR 30(15): 3433). A variety of vectors has been proposed for stable maintenance in cells based on S/MAR motifs, e.g. in U.S. Pat. No. 6,410,314 B1 and in Haase et al. (2010), BMC Biotechnology 10:20; moreover, epigenetic effects having an influence on replication of such vectors were identified (Haase et al. (2013), PLOS One 8(11):e79262).

Nonetheless, S/MAR-based vectors being stable enough for use in gene therapy are still not available.

There is, nonetheless, a need in the art for improved means and methods for stable transfection of cells, in particular using S/MAR elements while maintaining satisfactory expression of the transgene over extended periods of time. This problem is solved by the means and methods disclosed herein.

Accordingly, the present invention relates to a polynucleotide comprising at least one promoter, at least one expressible construct, and an S/MAR element, wherein said polynucleotide is an integration construct or a non-integrative vector construct, wherein said S/MAR element is located downstream of said promoter and of said expressible construct, and wherein said S/MAR element is flanked by a splice donor and a splice acceptor.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s). In the context of nucleic acid sequences, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “polynucleotide”, as used herein, refers to a linear or circular nucleic acid molecule. The term encompasses single as well as partially or completely double-stranded polynucleotides. Preferably, the polynucleotide is RNA or is DNA, including cDNA, more preferably is DNA. Moreover, comprised are also chemically modified polynucleotides including naturally occurring modified polynucleotides such as glycosylated or methylated polynucleotides or artificially modified derivatives such as biotinylated polynucleotides. The polynucleotide of the present invention shall be provided, preferably, either as an isolated polynucleotide (i.e. isolated from its natural context) or in genetically modified form. The polynucleotide of the invention comprises at least one promoter active in a host cell, at least one expressible construct, and an S/MAR element; moreover, the polynucleotide has the biological activity of providing expression of the expressible construct a host cell, all as specified herein below. Preferably, the polynucleotide has a length of at most 1 Mb, more preferably at most 500 kb, even more preferably at most 200 kb, most preferably at most 100 kb. Preferably, the polynucleotide is a non-naturally occurring polynucleotide; thus, preferably, the nucleotide is an artificial polynucleotide. Also preferably, the polynucleotide is a chimeric polynucleotide; more preferably, the polynucleotide comprises at least one nucleic acid sequence heterologous to the remaining nucleic acid sequences it comprises. Preferably, the polynucleotide is devoid of any centromere and/or telomere sequence. Also preferably the polynucleotide comprises a transcriptional insulator element upstream of said promoter. Preferred transcriptional insulator elements are element-40 and S/MAR elements as specified herein. Thus, preferably, the promoter and the expressible construct are insulated from the residual sequences comprised in the polynucleotide by the presence of at least one insulation element, more preferably by being flanked by insulation elements.

Preferably, the polynucleotide comprises further expression control sequences allowing expression of genes in prokaryotic and/or eukaryotic, preferably in eukaryotic host cells or isolated fractions thereof. Expression of said polynucleotide comprises transcription of the polynucleotide, preferably into a translatable mRNA. Regulatory elements ensuring expression in eukaryotic cells, preferably mammalian cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Examples for regulatory elements permitting expression in eukaryotic host cells are the AOX1 or GAL1 promoter in yeast or the SMVP-, U6-, H1-, 7SK-, CMV-EFS-, SV40-, or RSV-promoter (Rous sarcoma virus), CMV-enhancer, SV40-enhancer or a globin intron in mammalian and other animal cells. Regulatory sequences preferred for expression of miRNAs or siRNAs are also known in the art. Moreover, inducible or cell type-specific expression control sequences may be comprised in a polynucleotide of the present invention. Inducible expression control sequences may comprise tet or lac operator sequences or sequences inducible by heat shock or other environmental factors. Suitable expression control sequences are well known in the art. Besides elements which are responsible for the initiation of transcription, such regulatory elements may also comprise transcription termination signals, such as the SV40-poly-A site or the tk-poly-A site, downstream of the polynucleotide.

As used herein, the term polynucleotide, preferably, includes variants of the specifically indicated polynucleotides. More preferably, the term polynucleotide relates to the specific polynucleotides indicated. The term “polynucleotide variant”, as used herein, relates to a variant of a polynucleotide related to herein comprising a nucleic acid sequence characterized in that the sequence can be derived from the aforementioned specific nucleic acid sequence by at least one nucleotide substitution, addition and/or deletion, wherein the polynucleotide variant shall have the biological activity or activities as specified for the specific polynucleotide. Thus, it is to be understood that a polynucleotide variant as referred to in accordance with the present invention shall have a nucleic acid sequence which differs due to at least one nucleotide substitution, deletion and/or addition. Preferably, said polynucleotide variant comprises an ortholog, a paralog or another homolog of the specific polynucleotide or of a functional subsequence thereof, e.g. of an S/MAR element. Also preferably, said polynucleotide variant comprises a naturally occurring allele of the specific polynucleotide or of a functional subsequence thereof. Polynucleotide variants also encompass polynucleotides comprising a nucleic acid sequence which is capable of hybridizing to the aforementioned specific polynucleotides or functional subsequences thereof, preferably, under stringent hybridization conditions. These stringent conditions are known to the skilled worker and can be found in standard textbooks A preferred example for stringent hybridization conditions are hybridization conditions in 6× sodium chloride/sodium citrate (=SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1× to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined for example for a nucleic acid with approximately 100 bp (=base pairs) in length and a G+ C content of 50% in the absence of formamide; accordingly, other conditions more suitable for low-G+ C DNA, which are in principle known to the skilled person, may be found to be more appropriate by the skilled person. The skilled worker knows how to determine the hybridization conditions required by referring to standard textbooks. Alternatively, polynucleotide variants are obtainable by PCR-based techniques such as mixed oligonucleotide primer-based amplification of DNA, i.e. using degenerated primers against conserved domains of a polypeptide of the present invention. Conserved domains of a polypeptide may be identified by a sequence comparison of the nucleic acid sequence of the polynucleotide or the amino acid sequence of the polypeptide of the present invention with sequences of other organisms. As a template, DNA or cDNA from bacteria, fungi, plants or, preferably, from animals may be used. Further, variants include polynucleotides comprising nucleic acid sequences which are at least 70%, at least 75%, at least 80%, at least 85%, at least 90%, at least 95%, at least 98% or at least 99% identical to the specifically indicated nucleic acid sequences or functional subsequences thereof. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the amino acid sequences specifically indicated. The percent identity values are, preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution, 25, 351-360, 1987, Higgins et al., CABIOS, 5 1989: 151-153) or the programs Gap and BestFit (Needleman and Wunsch (J. Mol. Biol. 48; 443-453 (1970)) and Smith and Waterman (Adv. Appl. Math. 2; 482-489 (1981))), are preferably used. Preferably, said programs are used with their standard parameters. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

A polynucleotide comprising a fragment of any of the specifically indicated nucleic acid sequences, said polynucleotide retaining the indicated activity or activities, is also encompassed as a variant polynucleotide of the present invention. A fragment as meant herein, preferably, comprises at least 200, preferably at least 300, more preferably at least 400 consecutive nucleotides of any one of the specific nucleic acid sequences; or encodes an amino acid sequence comprising at least 100, preferably at least 200, more preferably at least 300 consecutive amino acids of any one of the specific amino acid sequences and still having the indicated activity.

The polynucleotides of the present invention either consist of, essentially consist of, or comprise the aforementioned nucleic acid sequences. Thus, they may contain further nucleic acid sequences as well. Specifically, the polynucleotides of the present invention may encode e.g. fusion proteins or selectable markers. Such fusion proteins may comprise as additional part polypeptides for monitoring expression (e.g., green, yellow, blue or red fluorescent proteins, alkaline phosphatase and the like) or so called “tags” which may serve as a detectable marker or as an auxiliary measure for purification purposes. Tags for the different purposes are well known in the art and are described elsewhere herein.

The polynucleotide comprises at least one expressible construct. The term “expressible construct”, as used herein, relates to a nucleic acid sequence of interest of being transferred into and expressed in a host cell. As used herein, the term expressible construct includes all nucleic acid sequences which causes at least one gene product to be produced in a host cell in the context of the polynucleotide as specified herein. Thus, the expressible construct, preferably, does not require a secondary promoter in addition to the one provided by the polynucleotide itself. It is however, also envisaged that the expressible construct comprises a secondary promoter as specified herein below, directing or additionally directing transcription of a sequence or of sequences of interest. Preferably, the expressible construct encodes more than one gene product; thus, the expressible construct may, e.g., encode two polypeptides, of which one may be a selectable marker as specified herein below. Preferably, the gene product is an RNA, including miRNA, siRNA, and mRNA, preferably, is an mRNA; and/or the gene product is a polypeptide as specified elsewhere herein. Thus, preferably, the expressible construct is a nucleic acid sequence encoding a polynucleotide, e.g. an RNA of interest and/or a polypeptide of interest.

Preferably, the RNA encoded is an interfering, non-coding nucleic acid. The non-coding interfering nucleic acid which is expressed from the polynucleotide, thus, may be typically an antisense RNA, siRNA, miRNA, or ribozyme. Thereby, gene expression in the host cell can be modified, i.e. down-regulated, by using the polynucleotide as specified herein, which may be used e.g. for treating diseases or disorders including those mentioned in this specification elsewhere. Due to the improved stability and expression characteristics of the polynucleotides specified herein, gene silencing approaches can be improved as well. Thus, preferably, the RNA of interest is a therapeutic RNA. As used herein, the term “therapeutic RNA” relates to any RNA mediating a change in a physiological and/or metabolic state of a host cell comprising said therapeutic RNA. Thus, preferably, a therapeutic RNA is in interfering, non-coding RNA a specified herein above, in particular a siRNA, a miRNA, an antisense RNA, or a ribozyme. Means and methods for designing interfering nucleic acids for use in e.g. gene silencing, are known to the skilled person. More preferably, a therapeutic RNA is an mRNA encoding a therapeutic polypeptide as specified herein below or a subunit or active fragment thereof. Preferably, the therapeutic RNA mediates a change in a physiological and/or metabolic state of a host cell comprising said therapeutic RNA, thereby contributing to amelioration or treatment of a disease or disorder, preferably as specified elsewhere herein.

The polypeptide of interest may, in principle, be any polypeptide overexpression of which in a host cell is desired. Preferred are polypeptides for which high and/or continued expression is desired. Preferably, the polypeptide of interest is a therapeutic polypeptide. As used herein, the term “therapeutic polypeptide” relates to any polypeptide mediating a change in a physiological and/or metabolic state of a host cell comprising said therapeutic polypeptide and/or of cells being in direct or in fluid contact with such host cell, preferably via a bodily fluid, more preferably via blood, lymph, saliva, cerebrospinal fluid, and/or interstitial fluid. More preferably, the therapeutic polypeptide is a polypeptide mediating a change in a physiological and/or metabolic state of a host cell and/or of cells being in direct or in fluid contact with such host cell, thereby contributing to amelioration or treatment of a disease or disorder, preferably as specified elsewhere herein. Preferably, the therapeutic polypeptide is an antibody, preferably as specified herein below. Also preferably, the therapeutic polypeptide is a T Cell Receptor (TCR), more preferably a human or chimeric T Cell receptor, a Chimeric Antigen Receptor (CAR), preferably MART1 TCR, or a polypeptide lacking in cells affected with a genetic disease as specified elsewhere herein. Thus, e.g. preferably, the polynucleotide comprises at least one expressible construct encoding a polypeptide providing phenylalanine-hydroxylase activity (EC 1.14.16.1) for treatment of phenylketonuria, or encoding the REP1 gene for treating Choroideremia, or encoding the RPE65 gene for treating Leber's congenital amorosis, or encoding Factors VIII, IX and/or X for treatment of Haemophilia, or encoding the USH2a gene for treating Ushers disease.

The term “antibody” is used herein in the broadest sense and specifically covers monoclonal antibodies, multispecific antibodies (e.g. bispecific antibodies) formed from at least two intact antibodies, single-chain antibodies, single-domain-antibodies (VHH), also known as nanobodies, and antibody fragments so long as they exhibit the desired binding activity. Preferably, the antibody is a single-chain antibody or a VHH (nanobody). Preferably, the antibody is a therapeutic antibody, i.e. has binding activity for a disease-related molecule, preferably a polypeptide, of therapeutic relevance and contributes to treatment of a disease or disorder caused or aggravated by said disease-related molecule. “Antibody fragments”, as relate to herein, comprise a portion of an intact antibody comprising the antigen-binding region thereof. Examples of antibody fragments include Fab, Fab′, F(ab′)2, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. “Fv” is the minimum antibody fragment which contains a complete antigen-binding site. Preferably, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three hypervariable regions (HVRs, also referred to as complementarity determining regions (CDRs)) of each variable domain interact to define an antigen-binding site. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site. The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 0 404 097; WO 1993/01161; Hudson et al., Nat. Med. 9 (2003) 129-134; and Hollinger et al., PNAS USA 90 (1993) 6444-6448. Triabodies and tetrabodies are also described in Hudson et al., Nat. Med. 9 (2003) 129-134. The term “single-domain antibody” (VHH) or “nanobody”, relates to an antibody fragment comprising one variable antibody domain and is, in principle, known to the skilled person. A review is provided, e.g. in Muyldermanns et al. (2009), Vet Immunol Immunopathol. 128(1-3):178. Preferably, the VHH comprises the CDRs of a heavy-chain antibody, preferably obtained from an alpaca, dromedar, camel, llama, or shark immunized with a target polypeptide. Preferably, the antibody is an anti-tumor antigen antibody, more preferably an anti-tumor specific antigen antibody, even more preferably an anti-carcinoembryonic antigen (CEA) antibody, still more preferably a single-chain anti-CEA antibody, most preferably encoded by SEQ ID NO:16.

Preferably, the expressible construct comprises or further comprises a coding sequence encoding a selectable marker polypeptide, preferably wherein the promoter of the polynucleotide and/or a secondary promoter and said selectable marker sequence together constitute a selectable marker gene. As used herein, the term “selectable marker sequence” is used as a shorthand for the expression “coding sequence encoding a selectable marker polypeptide”. The term “selectable marker” is in principle understood by the skilled person and relates to a nucleic acid sequence conferring, when expressed in a host cell, resistance to at least one condition mediating selective pressure to a host cell when applied thereto. Selectable markers are known in the art for prokaryotic and for eukaryotic cells. Preferably, the selectable marker is a selectable marker of an eukaryotic cell. Preferably, the selectable marker is a selectable marker polypeptide, more preferably a selectable marker polypeptide having transporter and/or enzymatic activity removing a selective compound from a host cell or modifying said selective compound to make it inactive. Preferably, the selectable marker gene further encodes at least one intron, preferably upstream of the sequence encoding the selectable marker polypeptide. Preferably, the selectable marker is a marker mediating resistance to puromycin, to blasticidin, neomycin, and/or to zeocin, more preferably to puromycin. Thus, preferably, the promoter and the selectable marker together constitute a puromycin resistance gene, a blasticidin resistance gene, a neomycin resistance gene, or a zeocin resistance gene, more preferably a puromycin resistance gene. Preferably, the selectable marker is a polypeptide providing resistance to a specific set of growth conditions, preferably presence and/or absence of proliferation signals. Thus, preferably, the selectable marker is a T-cell receptor (TCR) or a chimeric antigen receptor (CAR), both of which are in principle known in the art. Preferably, the TCR and/or the CAR have a known specificity, such that, preferably, T cell signaling can be induced in host cells comprising said TCR and/or CAR. Preferably, in such case, the host cell is a T cell or an NK cell. Preferably, the selectable marker gene is devoid of a poly-A signal and of transcription termination signal(s). Thus, preferably, the polynucleotide further comprises a coding sequence encoding a selectable marker (selectable marker sequence), said selectable marker sequence being comprised in the expressible construct, wherein preferably said promoter and/or a secondary promoter and said selectable marker sequence together constitute a selectable marker gene, and wherein said selectable marker is a selectable marker of a eukaryotic cell. Preferably the selectable marker is the puromycin acetyltransferase (Genbank Acc No. KX548903.1 (SEQ ID NO:9), encoded by nucleotides 535 to 1134 of Genbank Acc No. KX548903.1 (SEQ ID NO:10)). Thus, the selectable marker gene, preferably, comprises a nucleic acid sequence which a) causes expression of a puromycin resistance polypeptide comprising the sequence of SEQ ID NO:9; b) causes expression of a puromycin resistance polypeptide comprising a sequence at least 70% identical to the sequence of SEQ ID NO:9; c) comprises the sequence of SEQ ID NO:10; d) comprises a sequence at least 70% identical to the sequence of SEQ ID NO:10, e) comprises a nucleic acid sequence encoding a puromycin resistance polypeptide comprising, preferably consisting of, the sequence of SEQ ID NO:9, and/or f) comprises a nucleic acid sequence encoding a puromycin resistance polypeptide comprising, preferably consisting of, a sequence at least 70% identical to the sequence of SEQ ID NO:9.

As specified herein, the expressible construct intervenes the at least one promoter and the S/MAR element, wherein said S/MAR element is flanked by a splice donor and a splice acceptor; thus, preferably, the S/MAR element is spliced out from a transcript comprising the expressible construct. Splice donor and splice acceptor sites are known in the art. Preferably, the sequence encoding an expressible construct intervenes the at least one promoter and the S/MAR element, wherein said S/MAR element is flanked by a splice donor and a splice acceptor; thus, preferably, the S/MAR element is spliced out from a transcript encoding the polypeptide, preferably the therapeutic polypeptide.

Preferably, the expressible construct comprises a selectable marker and a sequence encoding an RNA or polypeptide of interest, preferably intervened by a sequence enabling expression of two (or more) polypeptides in a eukaryotic cell from one mRNA, e.g. an internal ribosomal entry sequence (IRES) or, more preferably, a self-cleaving peptide sequence such as, most preferably, a peptide 2A (P2A) sequence from porcine teschovirus-1. Appropriate sequences are known in the art, e.g. from Kim et al. (2011) PLoS ONE 6(4): e18556.

The term “host cell”, as used herein, relates to any cell capable of receiving, integrating or stably replicating the polynucleotide, and expressing the expressible construct. Preferably, the host cell is a eukaryotic cell, preferably a plant or yeast cell, e.g. a cell of a strain of baker's yeast, or is an animal cell. More preferably, the host cell is an insect cell or a mammalian cell, in particular a mouse or rat cell. Even more preferably, the host cell is a mammalian cell, most preferably is a human cell. Preferably, the host cell is a CD34+ Progenitor Cell; a CD61+ Thrombocyte; a CD19+ B-Lymphocyte; a CD14+ Monocyte; a CD15+ Granulocyte; a CD3+ Cytotoxic T-Lymphocyte, preferably also positive for CD8 and CD45; a CD3+ Helper T-Lymphocyte, preferably also positive for CD4 and CD45; a CD3+ activated T-Lymphocyte, preferably also positive for CD25 and CD45, a Tumor infiltrating Lymphocyte, or a Natural Killer (NK) cell. Also preferably, the host cell is an embryonic stem (ES) cell, an induced pluripotent stem cell (IPS) cell, an airway epithelial cell, a fibroblast, or a retinal epithelial cell. As will be understood by the skilled person, the polynucleotide may in addition have sequences permitting replication in a bacterial cell, in particular a bacterial origin of replication. Preferably, the bacterial cell is a cell of a laboratory strain of bacteria, more preferably an Escherichia coli cell.

The term “promoter” is, in principle, known to the skilled person as a genetic element directing, optionally in concert with further regulatory elements, the level of transcription of a given gene. A promoter may be constitutive, i.e. providing a constant level of transcription essentially independent of a host cell's state, or may be regulated, i.e. provide levels of transcription in dependence of a host cell's state. Moreover, a promoter may be cell type and/or tissue specific, i.e. provide a detectable level of transcription only in a few or only one cell type. Preferably, the promoter according to the present invention is active in the host cell as specified herein above. As will be understood by the skilled person, the selection of promoter may depend on the type of host cell intended for targeting; suitable promoters for specific cell types as well as constitutive promoters are known in the art. Preferably, the promoter is a eukaryotic promoter, more preferably a constitutive eukaryotic promoter, even more preferably a strong eukaryotic promoter. Preferably, the promoter is an EF1alpha (elongation factor 1 alpha) promoter, an UbiC (ubiquitin C) promoter, a ROSA 26 promoter, a PGK (phosphoglycerate kinase) promoter, and/or a CAG (chicken alpha-actin) promoter, more preferably is an EF1alpha promoter. Also preferably, the promoter is a cell- and/or tissue-specific eukaryotic promoter. As used herein, the term “promoter” is used for the promoter as specified above, whereas any other promoter potentially present on the polynucleotide in addition is referred to as “secondary promoter”. Thus, preferably, the promoter is a promoter directing transcription into the S/MAR sequence in a host cell; also preferably, a promoter not directing transcription into the S/MAR sequence of the polynucleotide, e.g. being a prokaryotic promoter, being transcriptionally insulated from the S/MAR sequence, and/or being a promoter directing transcription away from the S/MAR sequence, is a secondary promoter. Preferably, the promoter comprises less than 1000, more preferably less than 250, even more preferably less than 100, most preferably less than 20 contiguous base pairs corresponding to an Apolipoprotein B promoter; thus, preferably, the polynucleotide does not comprise a human Apolipoprotein B promoter, more preferably does not comprise an Apolipoprotein B promoter.

Preferably, the expressible construct is located immediately downstream of the promoter and/or the S/MAR sequence is located immediately downstream of the expressible construct. Preferably, being located “immediately downstream” is lacking an intervening transcription termination signal, more preferably is lacking an intervening gene. Thus, preferably, transcripts initiated at the promoter and including the expressible construct sequence preferably comprise a transcribed S/MAR sequence, more preferably comprise the complete S/MAR sequence comprised in the polynucleotide; as will be understood by the skilled person in view of the description elsewhere herein, the polynucleotide preferably further includes splicing sites mediating excision of the S/MAR sequence from the primary transcript; thus, more preferably, at least primary transcripts initiated at the promoter and including the expressible marker sequence preferably comprise a transcribed S/MAR sequence, more preferably comprise the complete S/MAR sequence comprised in the polynucleotide. Also preferably, the term “immediately downstream” includes a polynucleotide in which the promoter and the S/MAR sequence are separated by elongated nucleic acid sequences, provided a transcription termination signal is not intervening the promoter and the S/MAR. Preferably, the sequence intervening the promoter and the first nucleotide of the expressible construct, or the sequence intervening the last nucleotide of the expressible construct and the S/MAR sequence has a length of at most 2 kb, more preferably at most 0.5 kb, even more preferably at most 0.2 kb, still more preferably at most 0.1 kb, most preferably at most 50 bp.

The term “S/MAR element”, also known under the designation “scaffold/matrix attachment region”, is, in principle, known to the skilled person to relate to a DNA sequence mediating attachment of the nuclear matrix of a eukaryotic cell to said DNA. S/MAR sequences typically are derived from sequences in the DNA of eukaryotic chromosomes. A variety of S/MAR sequences is available, and sequences are available from public databases, e.g. as described in Liebich et al. (2002), Nucleic Acids Res. 30, 312-374. According to the present invention, the nucleic acid sequence of said S/MAR element (hitherto referred to as S/MAR sequence) preferably comprises at least 3 sequence motifs ATTA (SEQ ID NO:1) per 100 nucleotides over a stretch of at most 200 nucleotides. Thus, the motif comprised in the S/MAR sequence comprises a multitude of the four-nucleotide motif 5′-ATTA-3′. Preferably, the S/MAR sequence has a length of at least 200 nucleotide, more preferably at least 300 nucleotides, even more preferably at least 400 nucleotides, most preferably at least 500 nucleotides. Preferably, the S/MAR sequence has a length of at most 3 kb, more preferably at most 2 kb, even more preferably at most 1.5 kb, still more preferably at most 1 kb, even more preferably at most 0.5 kb, most preferably at most 0.25 kb. Thus, preferably, the S/MAR sequence has a length of from 0.2 kb to 3 kb, more preferably of from 0.3 kb to 2 kb, even more preferably of from 0.4 kb to 1.5 kb, most preferably of from 0.5 kb to 1 kb. As will be understood, the indication “comprises n sequence motifs per 100 nucleotides” relates to the average number of said sequence motifs calculated per 100 base pairs of sequence and, accordingly, may be a fraction number. E.g. the number of ATTA sequence motifs per 100 base pairs in SEQ ID NO:6 is 34/525 base pairs*100 base pairs=6.5. Preferably, the number of sequence motifs per 100 base pairs is determined over the whole length of the S/MAR sequence; in case of doubt, e.g. where a boundary of the S/MAR sequence cannot be determined, the number of sequence motifs per 100 base pairs of a polynucleotide, preferably, is the highest number determinable for any window of 200 bp within said polynucleotide, more preferably is the highest number determinable for any window of 500 bp within said polynucleotide. Preferably, the S/MAR sequence comprises at least 4 sequence motifs ATTA per 100 nucleotides over a stretch of at most 200 nucleotides, more preferably at least 5 sequence motifs ATTA per 100 nucleotides over a stretch of at most 200 nucleotides, still more preferably at least 6 sequence motifs ATTA per 100 nucleotides over a stretch of at most 200 nucleotides. Also preferably, the S/MAR sequence comprises at least 3 sequence motifs ATTA per 100 nucleotides over a stretch of at most 400 nucleotides, more preferably at least 4 sequence motifs ATTA per 100 nucleotides over a stretch of at most 400 nucleotides, even more preferably at least 5 sequence motifs ATTA per 100 nucleotides over a stretch of at most 400 nucleotides, most preferably at least 6 sequence motifs ATTA per 100 nucleotides over a stretch of at most 400 nucleotides. Also preferably, the S/MAR sequence comprises at least 3 sequence motifs ATTA per 100 nucleotides over a stretch of at most 500 nucleotides, more preferably at least 4 sequence motifs ATTA per 100 nucleotides over a stretch of at most 500 nucleotides, still more preferably at least 5 sequence motifs ATTA per 100 nucleotides over a stretch of at most 500 nucleotides, most preferably at least 6 sequence motifs ATTA per 100 nucleotides over a stretch of at most 500 nucleotides. Thus, preferably, the S/MAR sequence comprises at least 10 sequence motifs ATTA over a sequence of 500 nucleotides, more preferably at least 20 sequence motifs ATTA over a sequence of 500 nucleotides, still more preferably at least 30 sequence motifs ATTA over a sequence of 500 nucleotides. Preferably, at least 80%, more preferably at least 90%, most preferably at least 95% of the ATTA motifs in the S/MAR sequence are separated by of from 9 to 13, preferably by 10 to 12, most preferably by 11 base pairs, respectively.

Preferably, the S/MAR element comprises additional sequence motifs, preferably within the sequence comprising the ATTA motifs described herein above. Preferably, the sequence stretch of said S/MAR element comprising said ATTA sequence motifs further comprises at least one sequence motif ATTTA (SEQ ID NO:2), preferably at least 2 sequence motifs ATTTA, more preferably at least 4 sequence motifs ATTTA, most preferably at least 8 sequence motifs ATTTA. Also preferably, the sequence stretch of said S/MAR element comprising said ATTA sequence motifs and, optionally, said ATTTA motif(s), further comprises at least one, preferably at least two, more preferably at least four, most preferably at least six palindromic motifs, preferably motifs TAAATATTTTA (SEQ ID NO:3). Preferably, said motifs TAAATATTTTA are contiguous with at least one motif ATTA on the 5′ end and/or the 3′ end. Also preferably, the sequence stretch of the S/MAR element comprising said ATTA sequence motifs comprises at least one, preferably at least two, more preferably at least three, even more preferably at least four, most preferably at least five sequence motifs ATTATAAATATTTTAATTA (SEQ ID NO:4), more preferably sequence motifs ATTTAATTATAAATATTTTAATTA (SEQ ID NO:5).

Also preferably, the S/MAR sequence has a low G+ C content. The skilled person knows how to calculate the C+ G content of a known sequence by counting all guanine and cytidine bases in the sequence and dividing the cumulated result by the number of nucleotides in the sequence. Preferably, the sequence stretch of the S/MAR element comprising said sequence motifs ATTA has a G+ C content of at most 30%, more preferably at most 20%, still more preferably at most 15%, even more preferably at most 10%, most preferably at most 5%. Preferably, in cases where the boundary of an S/MAR element cannot be determined, the sequence used for calculation of the G+ C content is the same used for calculating the number of ATTA motifs per 100 base pairs, as specified herein above. Also preferably, the S/MAR sequence has a low number of CG dinucleotides. Preferably, the sequence stretch of said S/MAR element comprising said sequence motifs comprises at most 6 sequence motifs CG, more preferably at most 4, even more preferably at most 2, most preferably does not comprise a sequence motif CG.

Preferably, the S/MAR sequence comprises an S/MAR sequence of an Apolipoprotein B gene, preferably a human Apolipoprotein B gene, more preferably a 3'S/MAR sequence of a human Apolipoprotein B gene. More preferably, the S/MAR sequence comprises a variant of a human Apolipoprotein B gene, more preferably of a 3'S/MAR sequence of a human Apolipoprotein B gene. Thus, preferably, the S/MAR sequence comprises a sequence at least 70% identical to the sequence of SEQ ID NO:6, preferably of SEQ ID NO:7 or 8. More preferably, the S/MAR sequence comprises the nucleic acid sequence of SEQ ID NO:6, preferably of SEQ ID NO:7, more preferably SEQ ID NO:8. Preferably, the S/MAR sequence comprises a sequence at least 70% identical to the sequence of SEQ ID NO:15, more preferably the S/MAR sequence comprises the sequence of SEQ ID NO:15. Also preferably, the S/MAR sequence comprises an S/MAR sequence of a beta-interferon gene, preferably a human beta-interferon gene, more preferably an S/MAR sequence of a human beta-interferon gene. Thus, preferably, the S/MAR sequence comprises a sequence at least 70% identical to the sequence of SEQ ID NO:17; more preferably, the S/MAR sequence comprises the nucleic acid sequence of SEQ ID NO:17.

Preferably, the polynucleotide comprises a poly-A signal downstream of the S/MAR element: More preferably, the polynucleotide comprises a poly-A signal and a transcription termination signal downstream of the S/MAR element. The S/MAR element is flanked by a splice donor and a splice acceptor; thus, preferably, a transcript is transcribed from the promoter, from which transcript the sequence of the S/MAR element is spliced out. Also preferably, the S/MAR sequence preferably is spliced out of the transcript encoding the selectable marker after transcription. Also preferably, the polynucleotide further comprises a (secondary) bacterial origin of replication as specified herein above and/or a bacterial selectable marker gene. Preferably, the bacterial origin of replication and the promoter driving expression of the bacterial selectable marker gene are prokaryote-specific, i.e., more preferably, are non-functional in a host cell. Also preferably, the bacterial origin of replication and/or bacterial selectable marker gene, preferably all elements active in a prokaryotic cell comprised in the polynucleotide, is/are insulated from the residual sequences comprised in the polynucleotide by the presence of at least one insulation element, more preferably by being flanked by insulation elements. Preferably, the bacterial origin of replication and/or bacterial selectable marker gene, preferably all elements active in a prokaryotic cell, is/are insulated from the residual sequences comprised in the polynucleotide by the presence of at least one insulating element at the 5′ end and of at least one insulating element at the 3′ end. More preferably, the bacterial origin of replication and/or bacterial selectable marker gene, preferably all elements active in a prokaryotic cell comprised in the polynucleotide, is/are insulated from the promoter by the presence of at least one insulation element, more preferably by being flanked by insulation elements. Preferably, said insulation element(s) is(are) an anti-repressive element 40 element (SEQ ID NO:11) or a variant thereof and/or an S/MAR element.

Thus, preferably, the polynucleotide comprises the sequence of SEQ ID NO:7 or 8 or of a sequence at least 70% identical to the sequence of SEQ ID NO:7 or 8; preferably of SEQ ID NO:12 or of a sequence at least 70% identical to the sequence of SEQ ID NO:12, more preferably of SEQ ID NO:13 or of a sequence at least 70% identical to the sequence of SEQ ID NO:13, most preferably of SEQ ID NO:14 or of a sequence at least 70% identical to the sequence of SEQ ID NO:14. Preferably, the polynucleotide comprises the sequence of SEQ ID NO:14 with the nucleic acid sequence encoding GFP replaced by a nucleic acid sequence encoding a different polypeptide, preferably a therapeutic polypeptide, more preferably human T Cell Receptor (TCR), Chimeric Antigen Receptor (CAR), preferably MART1 TCR.

Preferably, the polynucleotide is an integration construct. As used herein, the term “integration construct” includes all polynucleotides having the activity of becoming covalently integrated into the genome of a host cell at a detectable rate when introduced into said host cell. Preferably, an integration construct is integrated at a rate of at least 100 integration events per fmol polynucleotide transfected, more preferably at least 1000 integration events per fmol polynucleotide transfected, even more preferably at least 10⁴ integration events per fmol polynucleotide transfected, most preferably at least 10⁵ integration events per fmol polynucleotide transfected. Preferably, the integration construct is devoid of a eukaryotic origin of replication, preferably is devoid of an origin of replication, more preferably is devoid of a nucleic acid sequence causing the polynucleotide to be replicated and stably maintained. Thus, preferably, the integration construct does not replicate episomally, preferably does not replicate autonomously, in a host cell, preferably in a mammalian cell. Preferably, the integration construct comprises at least one integration signal.

Integration signals are, in principle, known in the art, and include all kinds of signals inducing a host cell or a recombination system comprised therein to covalently integrate a polynucleotide comprising said integration signal into the genome of the host cell. Thus, preferably, the integration signal is a free terminus of a linear polynucleotide, preferably of a double-stranded polynucleotide, which may induce VD(J) recombination, or, preferably, if comprising suitable homologous sequences, homologous recombination. Thus, preferably, the polynucleotide as specified herein is a double-stranded, linear polynucleotide, preferably a double-stranded, linear DNA. Also preferably, the integration signal is recombinase recognition sequence, a viral integration signal, a transposable element, or the like. Thus, preferably, the integration signal is a cre or a lox recombination site, a lambda attachment site, a zinc finger recombinase recognition site, a Transcription Activator-Like Effector Nuclease (TALEN) recognition site, or a serine integrase recognition site, e.g. a recognition site for a PhiC31 integrase derived from Streptomyces phage φC31. Thus, preferably, the integration mediated by the integration signal may be non-sequence specific, essentially sequence specific, or sequence specific.

Also preferably, the polynucleotide is a non-integrative vector construct. The term “non-integrative vector construct”, as used herein, relates to a polynucleotide construct maintained for certain period of time in a host cell without being integrated into the host cell genome. Preferably, the non-integrative vector construct is detectable in a host cell after on average 50 cell divisions, more preferably after on average 100 cell divisions by methods known in the art or described elsewhere herein, preferably by PCR. as used herein, the term non-integrative vector construct relates to a polynucleotide comprising at least one nucleic acid sequence causing the polynucleotide to be replicated and stably maintained for at least the aforesaid period of time. As referred to herein, the at least one nucleic acid sequence causing the polynucleotide to be replicated and stably maintained is a sequence present in the polynucleotide in addition to the S/MAR sequence. Thus, preferably, the at least one nucleic acid sequence causing the polynucleotide to be replicated and stably maintained is not an S/MAR sequence. Preferably, the non-integrative vector construct is an artificial chromosome, preferably comprising a centromere and telomeres. Preferably, the non-integrative vector construct is a human artificial chromosome. More preferably, the non-integrative vector construct is a construct replicating episomally, i.e. in circular form. Thus, preferably, the non-integrative vector construct is a non-integrative viral vector construct.

The term “non-integrative viral vector construct”, as used herein, relates to a polynucleotide construct maintained for certain period of time, preferably as specified herein above, in a host cell without being integrated into the host cell genome and comprising a virus-derived replication signal. Thus, non-integrative viral vector constructs are, preferably, based on viruses modified to not contain an integrase activity. As an alternative, non-integrative viral vector constructs are, preferably, derived from viruses integrating into the genome of a host cell only at a very low frequency, preferably leading to less than 1 integration in 10⁵ infected cells, more preferably less than 1 integration in 10⁶ infected cells, still more preferably less than 1 integration in 10⁷ infected cells, most preferably less than 1 integration in 10⁸ infected cells. Thus, preferably, the non-integrative viral vector construct is a viral vector construct based on an adeno-associated virus (AAV), a herpesvirus, a simian virus 40 (SV40), or a papillomavirus. Preferably, the non-integrative viral vector construct is a non-integrative lentiviral construct.

As used herein, the term “replicating” relates to an activity of a polynucleotide to induce production of at least two replicas of said polynucleotide in a host cell during a cell replication cycle. Thus, preferably, replication of a polynucleotide in a host cell is determined by determining the presence of the polynucleotide after a series of cell divisions, in which a non-replicating polynucleotide would have been expected to be diluted out. Preferably, replication is stable replication, i.e. is replication to such an extent that the polynucleotide still is detectable in a host cell population after on average 50 cell divisions. Preferably, detection of a polynucleotide in a host cell population is performed by PCR under standard conditions.

The term “episomal” replication is, in principle, known to the skilled person to relate to replication of a polynucleotide without being integrated into the cellular genome, i.e. without becoming covalently integrated into the cellular genome. Thus, preferably, episomal replication of a polynucleotide is replication of said polynucleotide as an autonomous replication unit. Preferably, episomal replication is maintenance of the polynucleotide in the host cell in the form of a circularly closed double-stranded DNA molecule. As will be understood by the skilled person, the actual replication of said polynucleotide may involve other forms, e.g. in rolling circle replication. Episomal maintenance of circular DNA preferably is verified by the plasmid rescue procedure known to the skilled person; i.e. preferably, by preparing a lysate of host cells and transforming the DNA comprised therein into appropriate bacterial cells, e.g. E. coli cells; if a suitable number of bacterial colonies obtainable by said method comprises the circular DNA as a plasmid having the same restriction pattern and/or sequence as the original circular DNA, it is, preferably, assumed that the circular DNA was maintained episomally. A further method of verifying episomal maintenance, which is also known to the skilled person, is DNA/DNA blotting (“Southern Blot” method); thus, preferably, total DNA of host cells is prepared and digested with one or more restriction enzyme(s); if in a Southern Blot using the original plasmid as a probe only bands corresponding to the original circular DNA are visible, it is preferably concluded that the plasmid is maintained episomally. More preferably, episomal maintenance is verified as described herein in the Examples.

In accordance, the term “replicating episomally”, as used herein, relates to the activity of a polynucleotide to induce production of at least two replicas of said polynucleotide in a host cell during a cell replication cycle while said polynucleotide is present in said cell as an autonomously replicating entity; and stable episomal replication is episomal replication to such an extent that the polynucleotide is still detectable in the host cell after at least 50 cell divisions. Preferably, the aforesaid number of cell divisions is the average number of cell divisions for a population of cells.

Advantageously, it was found in the work underlying the present invention that by combining an S/MAR element flanked by splicing sites as specified with a promoter reading into an expressible construct and said S/MAR element, a polynucleotide is obtained which provides for high and long-lasting expression in host cells, even if the polynucleotide is integrated into the cellular genome or is maintained as a non-integrating viral vector.

The definitions made above apply mutatis mutandis to the following. Additional definitions and explanations made further below also apply for all embodiments described in this specification mutatis mutandis.

The present invention further relates to a composition comprising a polynucleotide according to the present invention.

The term “composition”, as used herein, as used herein, relates to a composition of matter comprising the compounds as specified and optionally one or more acceptable carrier(s). Preferably, the composition is a pharmaceutically acceptable composition; thus, preferably, the carrier is a pharmaceutically acceptable carrier. The compounds of the present invention can be formulated as, preferably pharmaceutically acceptable, salts. Preferred salts comprise acetate, methylester, HCl, sulfate, chloride and the like.

The carrier(s) must be acceptable in the sense of being compatible with the other ingredients of the formulation and being not deleterious to the recipient thereof. A carrier employed may be, for example, either a solid, a gel or a liquid. Exemplary of solid pharmaceutical carriers are lactose, terra alba, sucrose, talc, gelatin, agar, pectin, acacia, magnesium stearate, stearic acid and the like. Exemplary of liquid carriers are phosphate-buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax.

Said suitable carriers comprise those mentioned above and others well known in the art, see, e.g., Remington's Pharmaceutical Sciences, Mack Publishing Company, Easton, Pa. The diluent(s) is/are selected so as not to affect the biological activity of the compounds in the composition. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Preferably, the composition mediates entry of the polynucleotide into a host cell. Thus, preferably the composition comprises at least one transfection agent. The selection of an appropriate transfection agent may depend on the target host cell, as well as the specific application envisaged. Transfection agents, appropriate transfection conditions, as well as selection criteria therefor are well-known in the art. Also preferably, the composition comprises virus-like particles. Thus, preferably, the polynucleotide is packaged into virus-like particles, i.e. preferably, the polynucleotide is comprised in the virus-like particles; thus, more preferably, the composition comprises a genetically engineered viral particle comprising the polynucleotide as specified. Preferably, the virus-like particles or virus particles are derived from a virus as specified herein above.

Pharmaceutical compositions are, preferably, administered topically or systemically. Suitable routes of administration conventionally used for drug administration are oral, intravenous, or parenteral administration as well as inhalation. However, depending on the nature and mode of action of a compound, the pharmaceutical compositions may be administered by other routes as well. For example, polynucleotide compounds may be administered in a gene therapy approach by using viral vectors or viruses or liposomes, as specified herein above. Moreover, the compounds can be administered in combination with other drugs either in a common pharmaceutical composition or as separated pharmaceutical compositions wherein said separated pharmaceutical compositions may be provided in form of a kit of parts. The compounds are, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables.

A therapeutically effective dose of a pharmaceutical composition refers to an amount of the compounds to be used in a pharmaceutical composition of the present invention which prevents, ameliorates or treats the symptoms accompanying a disease or condition referred to in this specification. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. A typical dose can be, for example, in the range of 1 to 1000 μg; however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. However, depending on the subject and the mode of administration, the quantity of substance administration may vary over a wide range to provide from about 0.01 mg per kg body mass to about 10 mg per kg body mass. In case a viral vector, in particular adeno-associated viral vector is administered, preferred doses are from 5×10¹¹, to 2×10¹³ viral particles or viral genomes/kg body weight; as will be understood, these exemplary doses may be modified depending, in addition to the factors described above, on additional factors like type of virus, target organ, and the like.

The pharmaceutical compositions and formulations referred to herein are administered at least once in order to treat or ameliorate or prevent a disease or condition recited in this specification. However, the said pharmaceutical compositions may be administered more than one time, for example from one to four times daily up to a non-limited number of days.

Specific pharmaceutical compositions are prepared in a manner well known in the pharmaceutical art and comprise at least one active compound referred to herein above in admixture or otherwise associated with a pharmaceutically acceptable carrier or diluent. For making those specific pharmaceutical compositions, the active compound(s) will usually be mixed with a carrier or the diluent, or enclosed or encapsulated in a capsule, sachet, cachet, paper or other suitable containers or vehicles. The resulting formulations are to be adopted to the mode of administration, i.e. in the forms of tablets, capsules, suppositories, solutions, suspensions or the like. Dosage recommendations shall be indicated in the prescribers or users instructions in order to anticipate dose adjustments depending on the considered recipient.

The present invention also relates to a host cell comprising the polynucleotide according to the present invention, preferably integrated into its genome.

The present invention also relates to a polynucleotide according to the present invention, a composition according to the present invention, and/or a host cell according to the present invention, for use in medicine. The present invention further relates to a polynucleotide according to the present invention, a composition according to the present invention, and/or a host cell according to the present invention, for use in treating genetic disease.

The term “genetic disease”, as used herein, relates to a disease causally linked to one or more modifications, preferably mutations, in the genome of an individual. Thus, preferably, the genetic disease is causally linked to one or more epigenetic changes, more preferably is causally linked to one or more genetic mutations. As will be understood, symptoms of a genetic disease often are caused by expression of a mutated gene and/or lack of expression of a gene providing normal function of the gene product in one or more specific tissue(s) and/or cell type(s). Thus, it may be preferable to treat genetic disease only in those cells in which the mutation contributes to disease. Preferably, the genetic disease is a monogenic disease, i.e. is caused by a genetic alteration in one gene. More preferably, the genetic disease is a monogenic recessive disease, i.e. is caused by genetic alterations in both alleles of a gene; thus, preferably, the amelioration of symptoms is expected by provision of at least one unaltered copy of the affected gene. Most preferably, the genetic disease isphenylketonuria, alkaptonuria, Leber's Congenital Amaurosis, Choroideremia, Haemophilia, Ushers disease, or Stargardt disease. In a preferred embodiment, the genetic disease is cancer.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50% at least 60%, at least 70%, at least 80%, or at least 90% of the subjects of a given cohort or population.

The term “subject” relates to a metazoan organism. Preferably, the subject is an animal, more preferably a mammal, most preferably a human being. Preferably, the subject is suffering from a genetic disease as specified herein above.

The present invention further relates to a device comprising a polynucleotide according to the present invention, a composition according to the present invention, and/or a host cell according to the present invention.

The term “device”, as used herein relates to a system of means comprising at least the means operatively linked to each other as to allow administration of the compound or of the composition of the present invention. Preferred means for administering polynucleotides, compositions, or host cells are well known in the art. How to link the means in an operating manner will depend on the type of means included into the device and on the kind of administration envisaged. Preferably, the means are comprised by a single device in such a case. Said device may accordingly include a delivery unit for the administration of the compound or composition and a storage unit for storing said compound or composition until administration. However, it is also contemplated that the means of the current invention may appear as separate devices in such an embodiment and are, preferably, packaged together as a kit. The person skilled in the art will realize how to link the means without further ado. Preferred devices are those which can be applied without the particular knowledge of a specialized technician. In a preferred embodiment, the device is a syringe, more preferably with a needle, comprising the compound or composition of the invention. In another preferred embodiment, the device is an intravenous infusion (IV) equipment comprising the compound or composition. In another preferred embodiment, the device is an endoscopic device comprising the compound or medicament for flushing a site of administration, or further comprising a needle for topical application of the compound or composition, e.g. to a tumor. In still another preferred embodiment the device is an inhaler comprising the compound of the present invention, wherein, more preferably, said compound is formulated for administration as an aerosol.

The present application also relates to a method for stably expressing an expressible construct in a host cell, comprising

a) contacting said host cell with a polynucleotide according to the present invention and/or a composition according to the present invention, and,

b) thereby, stably expressing an expressible construct in a host cell.

The method for stably expressing an expressible construct in host cell of the present invention, preferably, is an in vitro method. Moreover, it may comprise steps in addition to those explicitly mentioned above. For example, further steps may relate, e.g., to providing a host cell or a sample comprising the same for step a), and/or applying selective pressure to the host cells contacted. Moreover, one or more of said steps may be performed by automated equipment.

The term “stably expressing” in a host cell is understood by the skilled person to relate to introducing a polynucleotide comprising an expressible construct, preferably a heterologous polynucleotide, into a cell such that the expressible construct is stably expressed by the host cell as specified herein above. Preferably, stable transfection does not comprise stable episomal replication of the polynucleotide. Preferably, stable expression comprises, after contacting, applying selective pressure to the host cell to select for the presence of a selectable marker. The selective pressure is applied after contacting, optionally excluding a first time frame allowing the polynucleotide to establish within the host cell; the duration of said first time frame allowing the polynucleotide to establish within the host cell will depend mostly on the type of host cell contacted and on the kind of selectable marker used; preferably, the duration of said first time frame allowing the polynucleotide to establish within the host cell is of from 1 h to 48 h, more preferably of from 2 h to 24, most preferably of from 3 h to 16 h. However, the duration of said first time frame allowing the polynucleotide to establish within the host cell may also be zero, i.e. selective pressure may be applied immediately after contacting or even during contacting. Selective pressure may be applied continuously, i.e. at essentially all time points after the first time frame allowing the polynucleotide to establish within the host cell, more preferably to prevent host cells not comprising the polynucleotide from proliferating; or it may be applied transiently, more preferably to remove cells not having received the polynucleotide. Preferably, transient application of selective pressure is used in cases where the polynucleotide is an integrating construct or in case cells are transferred back into an organism after said contacting. It is, however, also envisaged that no selective pressure is applied, in particular in cases where it is known that the efficiency of transfer of the polynucleotide into target host cells is sufficiently high and/or where a pure population of transgenic host cells is not of major importance. In a preferred embodiment, a stably transfected population of cells may also be obtained by allowing an expressible construct encoding a detectable polypeptide to be expressed as specified herein above, and electing cells expressing the cargo sequence, e.g. by cell sorting, preferably FACS.

The term “contacting”, as used in the context of the methods of the present invention, is understood by the skilled person. Preferably, the term relates to bringing at least one polynucleotide, vector, and/or host cell of the present invention in physical contact with a host cell, e.g. allowing the host cell and the compound(s) to interact. Preferably, contacting includes delivery of at least one polynucleotide of the present invention into the interior of a host cell, preferably via a delivery means as specified above.

The present invention also relates to a method for treating genetic disease in a subject, comprising

a) contacting said subject with a polynucleotide according to the present invention, a composition according to the present invention, and/or a host cell according to the present invention, and,

b) thereby, treating genetic disease in said subject.

The method for treating genetic disease of the present invention, preferably, is an in vivo method. Moreover, it may comprise steps in addition to those explicitly mentioned above. For example, further steps may relate, e.g., to providing a host cell or a sample comprising the same for step a), and/or re-administering said sample or host cell into the subject. Thus, the method for treating genetic disease, comprise the steps of the method for stably transfecting a host cell as specified above. Moreover, one or more of said steps may be performed by automated equipment.

Further, the present invention relates to a use of a polynucleotide of the present invention for stably genetically modifying a host cell.

Also, the present invention relates to a use of a polynucleotide according to the present invention, a composition according to the present invention, and/or a host cell according to the present invention, for the manufacture of a medicament. And to a use of a polynucleotide according to the present invention, a composition according to the present invention, and/or a host cell according to the present invention, for the manufacture of a medicament for treating genetic disease, preferably monogenic disease, more preferably monogenic recessive disease, most preferably F. In a preferred embodiment, the genetic disease is cancer, as specified herein above.

Also, the present invention relates to a use of a polynucleotide according to the present invention, a composition according to the present invention, and/or a host cell according to the present invention, for the genetic modification of a primary cell, preferably a primary dermal fibroblast, for the generation of an Induced Pluripotent Stem Cells (IPSCs). Preferably, said primary cell is a mouse or a human primary cell.

The term “primary cell” is understood by the skilled person as opposed to a cell of a cultured cell line; thus, preferably, a primary cell is a cell derived from a living organism and having been cultured for at most 20 passages, more preferably at most 15 passages, even more preferably at most 10 passages, still more preferably at most 5 passages. Most preferably, primary cells are cells derived directly from tissue of a living being, preferably a mouse or a human.

The term “stem cell” is also understood by the skilled person to relate to an un- or low-differentiated cell with the potential for differentiation into at least two cell types, preferably at least five cell types, more preferably at least one complete cell lineage. Preferably, the stem cell is a totipotent stem cell, more preferably a pluripotent stem cell. The term “Induced Pluripotent Stem Cell” or “IPSC” relates to a pluripotent stem cell derived from a differentiated cell, preferably a differentiated primary cell. Methods of generating IPSCs are known in the art and include, preferably, expression of four transcription factors in the cell (e.g from Takahashi et al. (2006), Cell. 126 (4):663).

The present invention also relates to a use of a polynucleotide according to the present invention, a composition according to the present invention, and/or a host cell according to the present invention, for the genetic modification of embryonic stem cells, preferably non-human embryonic stem cells. The present invention also relates to a use of a polynucleotide according to the present invention, a composition according to the present invention, and/or a host cell according to the present invention, for the manufacture of a medicament for treating genetic disease as specified herein above, preferably wherein said medicament comprises host cells comprising a polynucleotide of the present invention.

The present invention also relates to a use of a polynucleotide according to the present invention, a composition according to the present invention, and/or a host cell according to the present invention, for the genetic modification of stem cells for generating a transgenic animal, preferably non-human animal. The present invention further relates to a use of a polynucleotide according to the present invention, a composition according to the present invention, and/or a host cell according to the present invention, for the production of a transgenic animal, preferably non-human animal.

The term “transgenic animal” as used herein, relates to an animal comprising at least one heterologous polynucleotide, preferably introduced into said animal by methods of genetic engineering. Preferably, the transgenic animal comprises at least one, more preferably at least 10, still more preferably at least 1000, even more preferably at least 10000 cells comprising at least one polynucleotide according to the present invention.

Also, the present invention relates to a use of a polynucleotide according to the present invention, a composition according to the present invention, and/or a host cell according to the present invention, for the genetic modification of a single cell embryo, preferably a non-human single cell embryo, by pronuclear injection.

As is understood by the skilled person, the term “pronuclear injection” relates to injecting genetic material, preferably a polynucleotide of the present invention, into the nucleus of a fertilized oocyte, preferably to create a transgenic animal, preferably a non-human animal.

The present invention further relates to the use of the polynucleotide according to the present invention or the composition according to the present invention in modifying gene expression in a host cell.

The present invention also relates to a method for increasing expression of a eukaryotic expression construct comprising a promoter and an expressible construct, comprising including an S/MAR element flanked by a splice donor and a splice acceptor downstream of said expressible construct. The present invention also relates to a method for increasing expression of a eukaryotic expression construct comprising a promoter, an expressible construct, and an S/MAR element, comprising including a splice donor and a splice acceptor flanking said S/MAR element.

The present invention further relates to a use of an S/MAR element flanked by a splice donor and a splice acceptor for increasing expression of a eukaryotic expression construct. The present invention also relates to a use of a splice donor and a splice acceptor for increasing expression of a eukaryotic expression construct comprising an S/MAR element.

The methods and uses for increasing expression of a eukaryotic expression construct, preferably, are in vitro methods or uses. However, they may, preferably, also be in vivo method or uses, e.g. as part of a method of treatment. The methods and uses may comprise further steps in addition to those specifically mentioned. Additional steps may e.g. relate to providing host cells for which improved expression is desirable, providing a polynucleotide comprising said eukaryotic expression construct, preferably a polynucleotide of the present invention, contacting a host cell with said polynucleotide, incubating said host cell after said contacting, harvesting the product of said eukaryotic expression construct. In case the method or use is an in vivo method or use, it may comprise administrating or re-administrating the contacted cells to a subject.

The term “eukaryotic expression construct” is understood by the skilled person to relate to a polynucleotide comprising at least an expressible construct and a promoter, both as specified herein above, which cause the expressible construct to be expressed in a eukaryotic cell, preferably a host cell. Preferably, the eukaryotic expression construct comprises further expression control sequences as specified herein above, in particular a transcriptional terminator.

The term “increasing expression” is understood to relate to an increase of expression of a eukaryotic expression construct including the S/MAR element flanked by a splice donor and a splice acceptor compared to an expression construct including only the S/MAR element, i.e. an S/MAR element not flanked by a splice donor and a splice acceptor. Preferably, increasing expression is increasing transcription. More preferably, increasing expression is increasing production of a polypeptide by increasing transcription of an expressible construct comprising a sequence encoding said polypeptide.

In view of the above, the following embodiments are preferred:

1. A polynucleotide comprising at least one promoter, at least one expressible construct, and an S/MAR element, wherein said polynucleotide is an integration construct or a non-integrative vector construct, wherein said S/MAR element is located downstream of said promoter and of said expressible construct, and wherein said S/MAR element is flanked by a splice donor and a splice acceptor.

2. The polynucleotide of embodiment 1, wherein said polynucleotide is an integration construct or a non-integrative viral vector construct.

3. The polynucleotide of embodiment 1 or 2, wherein said polynucleotide is an integration construct.

4. The polynucleotide of any one of embodiments 1 to 3, wherein said integration construct comprises at least one integration signal.

5. The polynucleotide of embodiment 4, wherein said integration signal is a free terminus of a linear polynucleotide, a recombinase recognition sequence, a viral integration signal, or a transposable element.

6. The polynucleotide of any one of embodiments 1 to 5, wherein said polynucleotide is devoid of a eukaryotic origin of replication, preferably is devoid of an origin of replication.

7. The polynucleotide of any one of embodiments 1 to 6, wherein said polynucleotide does not replicate episomally in a host cell, preferably in a mammalian cell.

8. The polynucleotide of embodiment 1 or 3, wherein said polynucleotide is a non-integrative viral vector construct.

9. The polynucleotide of embodiment 8, wherein said non-integrative viral vector construct is a non-integrative lentivirus construct, an adeno-associated virus construct, a simian virus 40 construct, a papillomavirus construct, an adenovirus construct, a hepatitis virus construct, or a herpesvirus construct.

10. The polynucleotide of embodiment 8 or 9, wherein said polynucleotide replicates episomally in a host cell, preferably in a mammalian cell.

11. The polynucleotide of any one of embodiments 1 to 10, wherein said expressible construct comprises at least one coding sequence encoding a polypeptide, a sequence encoding a siRNA, a sequence encoding an miRNA, a sequence encoding an antisense RNA, and/or a sequence encoding a ribozyme.

12. The polynucleotide of any one of embodiments 1 to 11, wherein said polypeptide is a therapeutic polypeptide, preferably a human T Cell Receptor (TCR), Chimeric Antigen Receptor (CAR), preferably MART1 TCR.

13. The polynucleotide of any one of embodiments 1 to 12, wherein said expressible construct comprises at least one coding sequence encoding a selectable marker.

14. The polynucleotide of any one of embodiments 1 to 13, wherein said expressible construct comprises at least two coding sequences, preferably of which one encodes a selectable marker.

15. The polynucleotide of any one of embodiments 1 to 14, wherein said expressible construct comprises a coding sequence encoding a selectable marker (selectable marker sequence), wherein said promoter and said selectable marker sequence together constitute a selectable marker gene, and wherein said selectable marker is a selectable marker of a eukaryotic cell.

16. The polynucleotide embodiment 15, wherein said selectable marker gene is a puromycin resistance gene, a blasticidin resistance gene, a neomycin resistance gene, or a zeocin resistance gene, preferably is a puromycin resistance gene.

17. The polynucleotide of any one of embodiments 1 to 16, wherein a transcript is transcribed from said promoter, from which transcript the sequence of the S/MAR element is spliced out.

18. The polynucleotide of any one of embodiments 1 to 17, wherein a poly-A signal downstream of the S/MAR element is retained in said splicing.

19. The polynucleotide of any one of embodiments 1 to 18, wherein said host cell is a mammalian cell, preferably a human cell.

20. The polynucleotide of any one of embodiments 1 to 19, wherein said polynucleotide is devoid of any centromere and/or telomere sequence.

21. The polynucleotide of any one of embodiments 1 to 20, wherein said polynucleotide comprises a transcriptional insulator element upstream of said promoter.

22. The polynucleotide of embodiment 21, wherein said insulator element is an element-40 and/or an S/MAR element.

23. The polynucleotide of any one of embodiments 1 to 22, wherein said promoter and expressible construct are insulated from the residual sequences comprised in the polynucleotide by the presence of at least one insulation element, more preferably by being flanked by insulation elements.

24. A composition comprising a polynucleotide according to any one of embodiments 1 to 23.

25. A host cell comprising the polynucleotide according to any one of embodiments 1 to 23, preferably integrated into its genome.

26. The host cell of embodiment 25, wherein said host cell is a CD34+ Progenitor Cell; a CD61+ Thrombocyte; a CD19+ B-Lymphocyte; a CD14+ Monocyte; a CD15+ Granulocyte; a CD3+ Cytotoxic T-Lymphocyte, preferably also positive for CD8 and CD45; a CD3+ Helper T-Lymphocyte, preferably also positive for CD4 and CD45; a CD3+ activated T-Lymphocyte, preferably also positive for CD25 and CD45, a Tumor infiltrating Lymphocyte, a Natural Killer (NK) cell, an embryonic stem (ES) cell, an induced pluripotent stem cell (IPS) cell, an airway epithelial cell, a fibroblast, or a retinal epithelial cell.

27. The host cell of embodiment 25 or 26, wherein said polynucleotide is covalently bonded to a chromosome of said host cell.

28. A polynucleotide according to any one of embodiments 1 to 23, a composition according to embodiment 24, and/or a host cell according to any one of embodiments 25 to 27 for use in medicine,

29. A polynucleotide according to any one of embodiments 1 to 23, a composition according to embodiment 24, and/or a host cell according to any one of embodiments 25 to 27 for use in treating genetic disease.

30. A device comprising the polynucleotide according to any one of embodiments 1 to 23, the composition according to embodiment 24, and/or the host cell according to any one of embodiments 25 to 27

31. A method for stably expressing an expressible construct in a host cell, comprising

a) contacting said host cell with a polynucleotide according to any one of embodiments 1 to 23 and/or a composition according to embodiment 24, and,

b) thereby, stably expressing an expressible construct in a host cell.

32. A method for treating genetic disease in a subject, comprising

a) contacting said subject with a polynucleotide according to any one of embodiments 1 to 23, a composition according to embodiment 24, and/or a host cell according to any one of embodiments 25 to 27, and,

b) thereby, treating genetic disease in said subject.

33. Use of a polynucleotide according to any one of embodiments 1 to 23 and/or a composition according to embodiment 24, for stably genetically modifying a host cell.

34. Use of a polynucleotide according to any one of embodiments 1 to 23, a composition according to embodiment 24, and/or a host cell according to any one of embodiments 25 to 27, for stably genetically modifying a host cell.

35. Use of a polynucleotide according to any one of embodiments 1 to 23, a composition according to embodiment 24, and/or a host cell according to any one of embodiments 25 to 27 for the manufacture of a medicament.

36. Use of a polynucleotide according to any one of embodiments 1 to 23, a composition according to embodiment 24, and/or a host cell according to any one of embodiments 25 to 27 for the manufacture of a medicament for treating genetic disease, preferably monogenic disease, more preferably monogenic recessive disease, most preferably phenylketonuria, alkaptonuria, Leber's Congenital Amaurosis, Choroideremia, or Stargardt disease.

37. A method for increasing expression of a eukaryotic expression construct comprising a promoter and an expressible construct, comprising including an S/MAR element flanked by a splice donor and a splice acceptor downstream of said expressible construct.

38. The method of embodiment 37, wherein said eukaryotic expression construct further comprises a transcriptional terminator.

39. The method of embodiment, wherein said S/MAR element is included between said expressible construct and said transcriptional terminator.

40. The method of any one of embodiments 37 to 39, wherein said wherein said S/MAR element is spliced out of a primary transcript initiated at said promotor.

41. The method of any one of embodiments 37 to 40, wherein said method comprises providing a polynucleotide according to any one of embodiments 1 to 23 and/or a composition according to embodiment 24.

42. The method of any one of embodiments 37 to 41, wherein said method further comprises contacting a host cell with a polynucleotide comprising said expression construct, preferably a polynucleotide according to any one of embodiments 1 to 23 and/or a composition according to embodiment 24.

43. Use of an S/MAR element flanked by a splice donor and a splice acceptor for increasing expression of a eukaryotic expression construct.

44. The use of embodiment 43, wherein said eukaryotic expression construct comprises a promoter and an expressible construct.

45. The use of embodiment 44, wherein said use comprises including said S/MAR element downstream of said expressible construct.

46. The use of embodiment 44 or 45, wherein said eukaryotic expression construct further comprises a transcriptional terminator.

47. The use of embodiment 46, wherein said use comprises including said S/MAR element between said expressible construct and said transcriptional terminator.

48. The use of any one of embodiments 43 to 47, wherein said use comprises providing a polynucleotide according to any one of embodiments 1 to 23 and/or a composition according to embodiment 24.

49. The use of any one of embodiments 43 to 48, wherein said use further comprises contacting a host cell with a polynucleotide comprising said expression construct, preferably a polynucleotide according to any one of embodiments 1 to 23 and/or a composition according to embodiment 24.

All references cited in this specification are herewith incorporated by reference with respect to their entire disclosure content and the disclosure content specifically mentioned in this specification.

### FIGURE LEGENDS

FIG. 1: Schematic depiction of the expression cassettes. A) GFP-S/MAR vector. The expression cassette consists of a ubiquitous promoter (Promoter) that drives the expression of the reporter gene GFP and the selectable marker Puromycin divided by the self-cleavage sequence P2A. The expression cassette is followed by an S/MAR sequence and a transcriptional terminator (PolyA). B) The expression cassette consists of the same elements, but the S/MAR sequence is flanked by a splicing donor (SD) and a splicing acceptor (SA) site.

FIG. 2: Hek293T cell populations established with the GFP-S/MAR and the GFP-S/MARsplice vector were analysed for transgene expression 35 days post DNA delivery and selection in Puromycin (0.5 ug/ml) by Flow Cytometry (A). The relative expression of the transgene GFP was evaluated and normalised to the expression of the housekeeping gene GAPDH (B). The figures show that the introduction of splicing sequences improve the transgene expression by enhancing the RNA amount in the cells.

FIG. 3: Schematic depiction of transposon mediated expression cassettes. A) GFP-S/MAR vector. The expression cassette consists of a ubiquitous promoter (Promoter) that drives the expression of the reporter gene GFP and the selectable marker Puromycin divided by the self-cleavage sequence P2A. The 5′ and 3′ ITRs are the sequences that mediated the expression cassette transposition and integration into the cell target genome. The expression cassette is followed by an S/MAR sequence and a transcriptional terminator (PolyA). B) The expression cassette consists of the same elements, but the S/MAR sequence is flanked by a splicing donor (SD) and a splicing acceptor (SA) site.

FIG. 4: Colony forming assay (A) and analysis of the transgene expression in established cells (B). The efficiency of generating stably expressing cells was evaluated by a colony forming assay. Following DNA delivery into Hek293T, cells positive for GFP transgene expression were isolated via FACS sorting (FACS Aria II) and plated into a 6 cm cell culture dish. They were then cultured for 3 weeks in presence of 1 μg/ml Puromycin. After 3 weeks the cells were fixed with PFA, stained with Crystal Violet and counted. The number of colonies is considered as the efficiency of vector establishment. The generation of stable cells lines is significantly more effective with the construct number 2 where the MAR sequences is flanked by splicing sites (p<0.00001). The expression of transgene GFP was evaluated in established Heks293T as the fluorescent intensity of the cell populations. The expression of the reporter gene GFP is significantly higher (p<0.05) in Hek293T cells modified with the construct 2; 1=GFP-Puro-S/MAR, 2=GFP-Puro-S/MAR splice.

The following Examples shall merely illustrate the invention. They shall not be construed, whatsoever, to limit the scope of the invention.

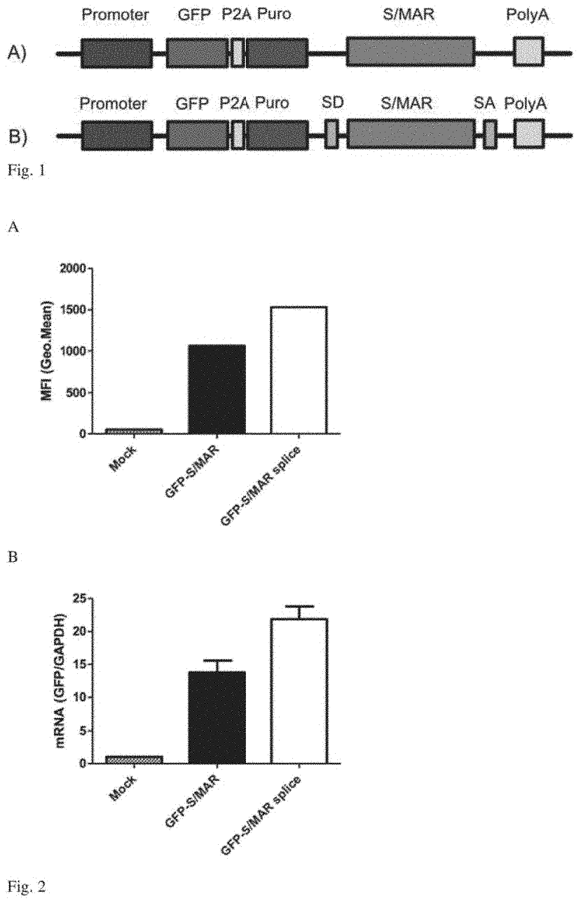

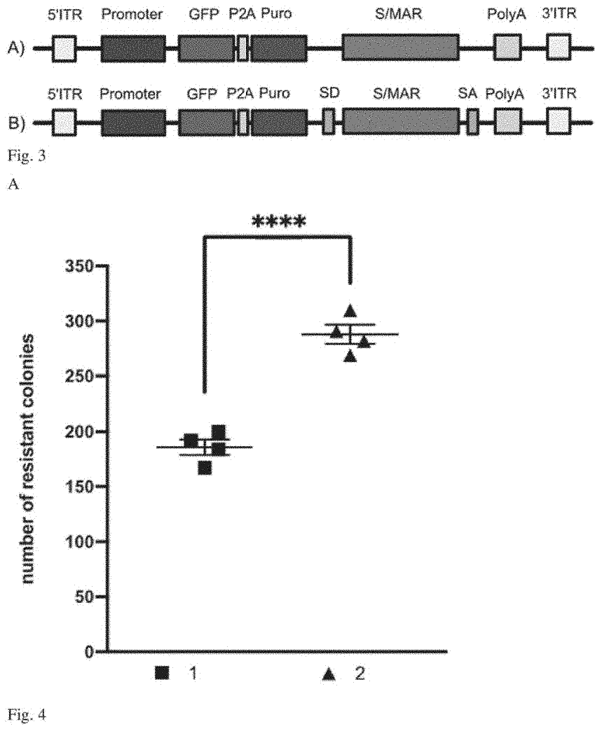

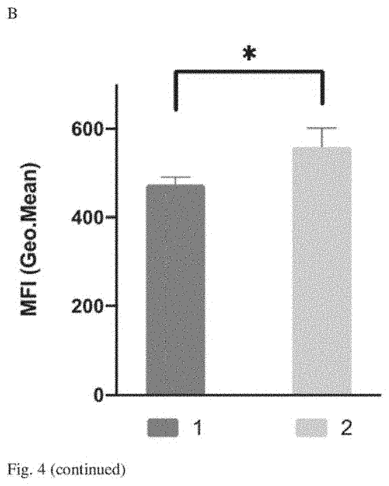

Example 1: Linear constructs as shown in FIG. 1 were purified and transfected into Hek293T cells. Cells were selected for 35 days in medium containing puromycin (0.5 μg/ml), whereafter relative GFP expression (amount of GFP RNA compared to GAPDH) and mean fluorescence intensity (MFI) in a FACS were determined. As shown in FIG. 2, transgene mRNA and protein levels are increased by the splicing sites flanking the S/MAR sequence.

1. A polynucleotide comprising at least one promoter, at least one expressible construct, and an S/MAR element, wherein said polynucleotide is an integration construct, wherein said S/MAR element is located downstream of said promoter and of said expressible construct, and wherein said S/MAR element is flanked by a splice donor and a splice acceptor.

2. The polynucleotide of claim 1, wherein said polynucleotide is an integration construct comprising at least one integration signal.

3. The polynucleotide of claim 1, wherein said integration signal is a free terminus of a linear polynucleotide, a viral integration signal, or a transposable element.

4. The polynucleotide of claim 1, wherein said expressible construct comprises at least one coding sequence encoding a polypeptide, a sequence encoding a siRNA, a sequence encoding an miRNA, a sequence encoding an antisense RNA, and/or a sequence encoding a ribozyme.

5. The polynucleotide of claim 4, wherein said polypeptide is a therapeutic polypeptide, preferably a human T Cell Receptor (TCR), Chimeric Antigen Receptor (CAR), preferably MART1 TCR.

6. The polynucleotide of claim 1, wherein a transcript is transcribed from said promoter, from which transcript the sequence of the S/MAR element is spliced out.

7. (canceled)

8. A host cell comprising the polynucleotide according to claim 1 integrated into its genome.

9. (canceled)

10. (canceled)

11. (canceled)

12. A method for increasing expression of a eukaryotic expression construct comprising a promoter and an expressible construct, the method comprising including an S/MAR element flanked by a splice donor and a splice acceptor downstream of said expressible construct, wherein said eukaryotic expression is an integration construct.

13. (canceled)

14. (canceled)

15. (canceled)

16. A method for treating genetic disease in a subject, comprising
a) contacting said subject with a polynucleotide according to claim 1, and
b) thereby, treating genetic disease in said subject.

17. The method of claim 16, wherein said polynucleotide is comprised in a host cell.

18. The method of claim 16, wherein said genetic disease is causally linked to one or more epigenetic changes and/or to one or more genetic mutations.

19. The method of claim 16, wherein the genetic disease is cancer, phenylketonuria, alkaptonuria, Leber's Congenital Amaurosis, Choroideremia, Haemophilia, Ushers disease, or Stargardt disease.


SEQUENCE LISTING (17 sequences)

SEQ ID NO: 1 - DNA, 4 bp, Homo sapiens
atta

SEQ ID NO: 2 - DNA, 5 bp, Homo sapiens
attta

SEQ ID NO: 3 - DNA, 11 bp, Homo sapiens
taaatatttta

SEQ ID NO: 4 - DNA, 19 bp, Homo sapiens
attataaatattttaatta

SEQ ID NO: 5 - DNA, 24 bp, Homo sapiens
atttaattataaatattttaatta

SEQ ID NO: 6 - DNA, 525 bp, Homo sapiens
atttataaaatattgaattataaaatatgtaattataaatactttaattataaaatatgtaattataaatactttaattataaaatatgtaattataaatactttataaaatatgtaattataaaatatgtaattataaacattttaattataaaatatgtaattataaacattttaattataaaatatgtaattataaacattttaattataaaatatgtaattataaacattttaattataaaatatgtaattataaacattttaattataaaatatttaattataaacattttaattataaaatatttaattataaatattttaattataaaatatttaattataaatattttaattataaaatatttaattataaatattttaattataaaatatttaattataaatactttaattataaaatatttaattataaatattttaattataaaatatttaattataaatattttaattataaatattttaattataaaatatttaattataaaaacacaatta

SEQ ID NO: 7 - DNA, 825 bp, Homo sapiens
gcaggctgagtgaaataaaggacttgttatttcatctcgaggcctaccggagagccttgccttgcaaaggcagacagtcagtgaggaagactatgtggcacatgaagacaccagaggtgttcctcaggatcaaagtatgtacaagcctttgtgaatattttttccttctcacttggcaaatacaattcctgagatcaataacctcgtctttttaattttttcctcgtctttttaactatttataaaatattgaattataaaatatgtaattataaatactttaattataaaatatgtaattataaatactttaattataaaatatgtaattataaatactttataaaatatgtaattataaaatatgtaattataaacattttaattataaaatatgtaattataaacattttaattataaaatatgtaattataaacattttaattataaaatatgtaattataaacattttaattataaaatatgtaattataaacattttaattataaaatatttaattataaacattttaattataaaatatttaattataaatattttaattataaaatatttaattataaatattttaattataaaatatttaattataaatattttaattataaaatatttaattataaatactttaattataaaatatttaattataaatattttaattataaaatatttaattataaatattttaattataaatattttaattataaaatatttaattataaaaacacaattacctcatctttttaaatatttttgcaaaatatttccctccataatttctccgtttccattttta

SEQ ID NO: 8 - DNA, 854 bp, Homo sapiens
cttctccactcctggcaggctgagtgaaataaaggacttgttatttcatctcgaggcctaccggagagccttgccttgcaaaggcagacagtcagtgaggaagactatgtggcacatgaagacaccagaggtgttcctcaggatcaaagtatgtacaagcctttgtgaatattttttccttctcacttggcaaatacaattcctgagatcaataacctcgtctttttaattttttcctcgtctttttaactatttataaaatattgaattataaaatatgtaattataaatactttaattataaaatatgtaattataaatactttaattataaaatatgtaattataaatactttataaaatatgtaattataaaatatgtaattataaacattttaattataaaatatgtaattataaacattttaattataaaatatgtaattataaacattttaattataaaatatgtaattataaacattttaattataaaatatgtaattataaacattttaattataaaatatttaattataaacattttaattataaaatatttaattataaatattttaattataaaatatttaattataaatattttaattataaaatatttaattataaatattttaattataaaatatttaattataaatactttaattataaaatatttaattataaatattttaattataaaatatttaattataaatattttaattataaatattttaattataaaatatttaattataaaaacacaattacctcatctttttaaatatttttgcaaaatatttccctccataatttctccgtttccatttttattctgttacttaaat

SEQ ID NO: 9 - protein, 199 aa, Artificial
  puromycin acetyltransferase, synthetic construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 10 - DNA, 600 bp, Artificial
  puromycin acetyltransferase encoding sequence, synthetic construct
atgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 11 - DNA, 1031 bp, Artificial
  anti-repressive element40
gatcaagaaagcactccgggctccagaaggagccttccaggccagctttgagcataagctgctgatgagcagtgagtgtcttgagtagtgttcagggcagcatgttaccattcatgcttgacttctagccagtgtgacgagaggctggagtcaggtctctagagagttgagcagctccagccttagatctcccagtcttatgcggtgtgcccattcgctttgtgtctgcagtcccctggccacacccagtaacagttctgggatctatgggagtagcttccttagtgagctttcccttcaaatactttgcaaccaggtagagaagtttggagtgaaggttttgttcttcgtttcttcacaatatggatatgcatcttcttttgaaaatgttaaagtaaattacctctcttttcagatactgtcttcatgcgaacttggtatcctgtttccatcccagccttctataacccagtaacatcttttttgaaaccagtgggtgagaaagacacctggtcaggaacgcggaccacaggacaactcaggctcacccacggcatcagactaaaggcaaacaaggactctgtataaagtaccggtggcatgtgtattagtggagatgcagcctgtgctctgcagacagggagtcacacagacacttttctataatttcttaagtgctttgaatgttcaagtagaaagtctaacattaaatttgattgaacaattgtatattcatggaatattttggaacggaataccaaaaaatggcaatagtggttctttctggatggaagacaaacttttcttctttaaaataaattttattttatatatttgaggttgaccacatgaccttaaggatacatatagacagtaaactggttactacagtgaagcaaattaacatatctaccatcgtacatagttacatttttttgtgtgacaggaacagctaaaatctacgtatttaacaaaactcctaaagacaatacatttttattaactatagccctcatgatgtacattagatc

SEQ ID NO: 12 - DNA, 1438 bp, Artificial
  CMV Promoter - S/MAR sequence
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcttctccactcctggcaggctgagtgaaataaaggacttgttatttcatctcgaggcctaccggagagccttgccttgcaaaggcagacagtcagtgaggaagactatgtggcacatgaagacaccagaggtgttcctcaggatcaaagtatgtacaagcctttgtgaatattttttccttctcacttggcaaatacaattcctgagatcaataacctcgtctttttaattttttcctcgtctttttaactatttataaaatattgaattataaaatatgtaattataaatactttaattataaaatatgtaattataaatactttaattataaaatatgtaattataaatactttataaaatatgtaattataaaatatgtaattataaacattttaattataaaatatgtaattataaacattttaattataaaatatgtaattataaacattttaattataaaatatgtaattataaacattttaattataaaatatgtaattataaacattttaattataaaatatttaattataaacattttaattataaaatatttaattataaatattttaattataaaatatttaattataaatattttaattataaaatatttaattataaatattttaattataaaatatttaattataaatactttaattataaaatatttaattataaatattttaattataaaatatttaattataaatattttaattataaatattttaattataaaatatttaattataaaaacacaattacctcatctttttaaatatttttgcaaaatatttccctccataatttctccgtttccatttttattctgttacttaaat

SEQ ID NO: 13 - DNA, 2038 bp, Artificial
  CMV Promoter - Puromycin - S/MAR sequence
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacttctccactcctggcaggctgagtgaaataaaggacttgttatttcatctcgaggcctaccggagagccttgccttgcaaaggcagacagtcagtgaggaagactatgtggcacatgaagacaccagaggtgttcctcaggatcaaagtatgtacaagcctttgtgaatattttttccttctcacttggcaaatacaattcctgagatcaataacctcgtctttttaattttttcctcgtctttttaactatttataaaatattgaattataaaatatgtaattataaatactttaattataaaatatgtaattataaatactttaattataaaatatgtaattataaatactttataaaatatgtaattataaaatatgtaattataaacattttaattataaaatatgtaattataaacattttaattataaaatatgtaattataaacattttaattataaaatatgtaattataaacattttaattataaaatatgtaattataaacattttaattataaaatatttaattataaacattttaattataaaatatttaattataaatattttaattataaaatatttaattataaatattttaattataaaatatttaattataaatattttaattataaaatatttaattataaatactttaattataaaatatttaattataaatattttaattataaaatatttaattataaatattttaattataaatattttaattataaaatatttaattataaaaacacaattacctcatctttttaaatatttttgcaaaatatttccctccataatttctccgtttccatttttattctgttacttaaat

SEQ ID NO: 14 - DNA, 6189 bp, Artificial
  Element40-GPF-P2A-Puromycin-S/MAR
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagcgctaccggtcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctggcagcggcgccaccaacttcagcctgctgaagcaggccggcgacgtggaggaaaaccctgggcccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaagatctatgcatgcagaagttggtcgtgaggcactgggcaggtaagtatcaaggttacaagacaggtcgacttctccactcctggcaggctgagtgaaataaaggacttgttatttcatctcgaggcctaccggagagccttgccttgcaaaggcagacagtcagtgaggaagactatgtggcacatgaagacaccagaggtgttcctcaggatcaaagtatgtacaagcctttgtgaatattttttccttctcacttggcaaatacaattcctgagatcaataacctcgtctttttaattttttcctcgtctttttaactatttataaaatattgaattataaaatatgtaattataaatactttaattataaaatatgtaattataaatactttaattataaaatatgtaattataaatactttataaaatatgtaattataaaatatgtaattataaacattttaattataaaatatgtaattataaacattttaattataaaatatgtaattataaacattttaattataaaatatgtaattataaacattttaattataaaatatgtaattataaacattttaattataaaatatttaattataaacattttaattataaaatatttaattataaatattttaattataaaatatttaattataaatattttaattataaaatatttaattataaatattttaattataaaatatttaattataaatactttaattataaaatatttaattataaatattttaattataaaatatttaattataaatattttaattataaatattttaattataaaatatttaattataaaaacacaattacctcatctttttaaatatttttgcaaaatatttccctccataatttctccgtttccatttttattctgttacttaaataattctgcagtcgacggttactgacatccactttgcctttctctccacaggtgtccactctaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttacgggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgccgtctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctctctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagtacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcataatattattgaagcatttatcagggttcgtctcgtcccggtctcctcccatgcatgtcaatattggccattagccatattattcattggttatatagcataaatcaatattggctattggccattgcatacgttgtatctatatcataatatgtacaaagcttgatcaagaaagcactccgggctccagaaggagccttccaggccagctttgagcataagctgctgatgagcagtgagtgtcttgagtagtgttcagggcagcatgttaccattcatgcttgacttctagccagtgtgacgagaggctggagtcaggtctctagagagttgagcagctccagccttagatctcccagtcttatgcggtgtgcccattcgctttgtgtctgcagtcccctggccacacccagtaacagttctgggatctatgggagtagcttccttagtgagctttcccttcaaatactttgcaaccaggtagagaagtttggagtgaaggttttgttcttcgtttcttcacaatatggatatgcatcttcttttgaaaatgttaaagtaaattacctctcttttcagatactgtcttcatgcgaacttggtatcctgtttccatcccagccttctataacccagtaacatcttttttgaaaccagtgggtgagaaagacacctggtcaggaacgcggaccacaggacaactcaggctcacccacggcatcagactaaaggcaaacaaggactctgtataaagtaccggtggcatgtgtattagtggagatgcagcctgtgctctgcagacagggagtcacacagacacttttctataatttcttaagtgctttgaatgttcaagtagaaagtctaacattaaatttgattgaacaattgtatattcatggaatattttggaacggaataccaaaaaatggcaatagtggttctttctggatggaagacaaacttttcttctttaaaataaattttattttatatatttgaggttgaccacatgaccttaaggatacatatagacagtaaactggttactacagtgaagcaaattaacatatctaccatcgtacatagttacatttttttgtgtgacaggaacagctaaaatctacgtatttaacaaaactcctaaagacaatacatttttattaactatagccctcatgatgtacattagatctgtacatttatattggctcatgtccaatatgaccgccatgtt

SEQ ID NO: 15 - DNA, 248 bp, Homo sapiens
atttaattataaacattttaattataaaatatttaattataaatattttaattataaaatatttaattataaatattttaattataaaatatttaattataaatattttaattataaaatatttaattataaatactttaattataaaatatttaattataaatattttaattataaaatatttaattataaatattttaattataaatattttaattataaaatatttaattataaaaacacaatta

SEQ ID NO: 16 - DNA, 777 bp, Artificial
  anti-CEA single-chain antibody
atggattttcaggtgcagattttcagcttcctgctaatcagtgcctcagtcataatgtctagaggtgtacactccgactacaaagacgagcaggtccaactgcaggagtcaggacctgacctggtgaaaccttctcagtcactttcactgacttgcactgtcactggctactccatcaccagtggttatagctggcactggattcggcagtttccaggaaacaaactggaatggatgggctacatacaatacagtggtatcactaactacaacccctctctcaaaagtcgaatctctatcacccgagacacatccaagaaccagttcttcctgcagttgaattctgtgactactgaggacacagccacatattactgtgcaagagaagactatgattaccactggtacttcgatgtctggggccaagggaccacggtcaccgtctcctcaggaggtggtggatcgggcggtggcgggtcgggtggcggcggatctgacatccagctgacccagtctccagcaatcatgtctgcatctctaggggaggagatcaccctaacctgcagtgccagctcgagtgtaagttacatgcactggtaccagcagaagtcaggcacttctcccaaactcttgatttatagcacatccaacctggcttctggagtcccttctcgcttcagtggcagtgggtctgggaccttttattctctcacaatcagcagtgtggaggctgaagatgctgccgattattactgccatcagtggagtagttatccc

SEQ ID NO: 17 - DNA, 1990 bp, Homo sapiens
agatctaaataaacttataaattgtgagagaaattaatgaatgtctaagttaatgcagaaacggagagacatactatattcatgaactaaaagacttaatattgtgaaggtatactttctttccacataaatttgtagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatgatctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcatgacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcaatattgatggtatataaccttgatatgatgtgatgagaacagtactttaccccatgggcttcctccccaaacccttaccccagtataaatcatgacaaatatactttaaaaaccattaccctatatctaaccagtactcctcaaaactgtcaaggtcatcaaaaataagaaaagtctgaggaactgtcaaaactaagaggaacccaaggagacatgagaattatatgtaatgtggcattctgaatgagatcccagaacagaaaaagaacagtagctaaaaaactaatgaaatataaataaagtttgaactttagttttttttaaaaaagagtagcattaacacggcaaagccattttcatatttttcttgaacattaagtacaagtctataattaaaaattttttaaatgtagtctggaacattgccagaaacagaagtacagcagctatctgtgctgtcgcctaactatccatagctgattggtctaaaatgagatacatcaacgctcctccatgttttttgttttctttttaaatgaaaaactttattttttaagaggagtttcaggttcatagcaaaattgagaggaaggtacattcaagctgaggaagttttcctctattcctagtttactgagagattgcatcatgaatgggtgttaaattttgtcaaatgctttttctgtgtctatcaatatgaccatgtgattttcttctttaacctgttgatgggacaaattacgttaattgattttcaaacgttgaaccacccttacatatctggaataaattctacttggttgtggtgtatattttttgatacattcttggattctttttgctaatattttgttgaaaatgtttgtatctttgttcatgagagatattggtctgttgttttcttttcttgtaatgtcattttctagttccggtattaaggtaatgctggcctagttgaatgatttaggaagtattccctctgcttctgtcttctgaaagagattgtagaaagttgatacaatttttttttctttaaatatcttgatagaattc